(19) United States
(12) Patent Application Publication
(10) Pub. No.: US 2024/0385175 A1
(43) Pub. Date: Nov. 21, 2024

(54) NON-INVASIVE SITE-SPECIFIC MEASUREMENT OF TRANSGENE EXPRESSION IN THE BRAIN

(71) Applicant: William Marsh Rice University, Houston, TX (US)

(72) Inventors: Jerzy Olgierd Szablowski, Houston, TX (US); Joon Pyung Seo, Houston, TX (US)

(21) Appl. No.: 18/667,652

(22) Filed: May 17, 2024

Related U.S. Application Data
(60) Provisional application No. 63/467,522, filed on May 18, 2023.

(51) Int. Cl. G01N 33/50; C12N 15/85; G01N 33/68

(52) U.S. Cl. CPC G01N 33/5023; C12N 15/85; G01N 33/6896; G01N 2800/28

(57) ABSTRACT

The use of synthetic serum markers to monitor transgene expression in specific brain regions through ultrasound-triggered release is described. In particular, the disclosed techniques relate to non-invasively measuring gene expression at specific sites in the brain. These techniques use a brain blood barrier (BBB) permeable, brain-specific AAV (PHP.eB) to deliver and express an engineered secreted protein marker in neurons. Focused ultrasound insonation opens the blood-brain barrier at specific targeted brain regions, which enables marker proteins to release into the blood for subsequent non-invasive detections. These techniques allow for the detection of localized gene activity in deep brain regions without tissue destruction.

### CROSS REFERENCE TO RELATED APPLICATIONS

This application claims priority from and the benefit of U.S. Provisional Application Ser. No. 63/467,522, entitled “NONINVASIVE SITE-SPECIFIC MEASUREMENT OF TRANSGENE EXPRESSION IN THE BRAIN”, filed May 18, 2023, which is hereby incorporated by reference in its entirety for all purposes.

### BACKGROUND

Monitoring gene expression in the living brain is useful for studying its network activity, diagnosing neurological diseases from onset to progression, and translating gene therapies into the clinic. For example, formation of memories requires activation of gene expression. Gene expression is also implicated in pathogenesis of Alzheimer's disease. Finally, establishing the success of gene therapy delivery is necessary to understand the gene delivery efficacy, durability, and site-specificity. However, mapping brain gene expression poses significant challenges, especially within the brain.

Typically measuring gene expression in the brain occurs through tissue-destructive techniques such as biopsy and post-mortem histology. Although common, these approaches are invasive, prohibit repeated measurements, and thus preclude longitudinal assessment of the same subject. Further, they hamper translational and neuroscientific studies, where damage to the brain region being investigated introduces a scientific confound and poses ethical challenges.

In consequence, several non-invasive imaging tools have emerged to detect endogenous gene expression within the brain. These include magnetic resonance imaging (MRI) with genetically encoded contrast agents, positron emission tomography (PET) using transgene-binding probes, optical imaging, and biomolecular ultrasound. These tools are transformative, but also must contend with significant barriers, including sensitivity constraints, the need for brain-delivered radioactive probes, poor penetration in deep tissues, and signal attenuation through the skull, respectively. Because these techniques rely on penetrant forms of energy to monitor gene expression, they also lack the ability to distinguish large numbers of different signals at once, in contrast to sensitive proteomic or transcriptomic approaches which can measure millions of molecular species in one sample. Overall, these obstacles have not addressed the need to measure non-invasively and with spatial precision the endogenous gene expression of critical neuronal activity markers, such as immediate early gene c-Fos, or expression of multiple genes at once. More sensitive techniques like intravital imaging with luciferases can measure c-Fos activity in small animals, but light generated by luciferases is subject to tissue scattering and skull attenuation which limits their utility in deep brain regions or large animals and prevents accurate localization of the source.

Recently, focused ultrasound-mediated blood-brain barrier opening (FUS-BBBO) was shown to release proteins from the brain into the circulation, in a process called FUS liquid biopsy. This process can measure molecular components within the intact brain tissue, but can only release naturally-existing tissue markers. Unfortunately, many gene products are not released into the brain in sufficient quantities to be detectable in the blood following FUS liquid biopsy. To address the issues with limited numbers and sensitivities of natural serum markers, synthetic serum markers have been developed that can report on transgene expression within the brain with a simple blood test. In such methods, engineered markers are expressed within the brain, but can cross through the intact blood-brain barrier (BBB) into the bloodstream using the process of reverse transcytosis. These markers, called Released Markers of Activity (RMA), have been shown to originate from the genetically-labeled cells in the brain and can inform on transduction or endogenous promoter activity. Because RMAs are engineered to be highly sensitive, as few as ˜12±9 cells in the brain need to express RMAs to make them detectable in blood. Because RMAs are proteins, they can be detected in multiplex using any protein chemistry techniques. The RMA approach, however, has two limitations. To achieve spatial precision of monitoring with RMAs, one needs to deliver the genes encoding RMAs to specific brain regions. The simplest way to achieve this is through a direct intracranial injection, which is invasive. Non-invasive gene delivery can be done with BBB-permeable AAVs (or other suitable vectors including, but not limited to: mRNA lipids nanoparticles (LNPs), lentivirus, or other synthetic nanoparticles) that are injected intravenously, but transduce cells throughout the brain, without spatial precision. Non-invasive gene delivery to specific brain regions can also be achieved with FUS-BBBO, but it also requires systemic delivery of AAVs. This results in a second limitation—introduction of a potential for non-brain tissues to release markers into the blood, confounding the readout.

This invention was funded in part by the Robert A. Welch Foundation under Welch Grant No. C-2048.

### BRIEF DESCRIPTION

Existing techniques for determining whether gene therapy is retained in the brain are insufficient. The techniques discussed herein allow monitoring of the presence of gene therapy in the brain with a simple insonation followed by a blood test. As such, it can be used to monitor the location of gene delivery with focused ultrasound, and the efficiency of delivery with testing blood. To achieve this, we combine synthetic markers that are expressed from the brain cells and deposited into the interstitial space. We then use focused ultrasound to open the BBB and release those interstitial markers from the brain into the blood so they can be easily measured. The total amount of markers released into the blood is correlated with the amount of markers present in the brain, which allows us to confirm presence or lack of gene therapy in the brain.

Gene expression is a critical component of brain physiology, but monitoring this expression in the living brain represents a significant challenge. Here, a new paradigm is described called Recovery of Markers through InSonation (REMIS) for non-invasive measurement of gene expression in the brain with cell-type, spatial, and temporal specificity. This approach relies on engineered protein markers that are produced in neurons but exit into the brain's interstitium. When ultrasound is applied to targeted brain regions, it opens the blood-brain barrier and releases these markers into the bloodstream. Once in blood, the markers can be readily detected using biochemical techniques. REMIS can non-invasively confirm gene delivery and measure endogenous signaling in specific brain sites through a simple insonation and a subsequent blood test. REMIS is reliable and demonstrated consistent improvement in recovery of markers from the brain into the blood. Overall, this work establishes a non-invasive, spatially-specific method of monitoring gene delivery and endogenous signaling in the brain. Further, the presently described technique resolves limitations of prior techniques and provides a non-invasive method of monitoring gene expression in specific brain regions after non-invasive gene delivery.

As discussed herein, with the described approach, designer markers are expressed within the brain in response to gene activity, and then released from targeted brain regions into the blood via focused ultrasound application. The presently described approach incorporates FUS-BBBO to non-invasively target specific brain regions with millimeter precision, enabling the transport of synthetic markers from the brain into blood. From there, the markers can be detected using any blood test capable of detecting the expressed proteins including, but not limited to: single molecule array for protein detection (SIMOA), enzymatic activity assays specific to the test protein, SDS-page, western blot, next-generation protein sequencing, biochemical assays such as the enzyme-linked immunosorbent assay (ELISA), in vivo detection of luciferases, surface plasmon resonance (SPR), mass spectrometry, or any other suitable technique for detecting and/or quantifying a target protein within a blood sample. In this manner routine measurements of gene expression from blood draws may be facilitated.

To establish REMIS, markers were first expressed in neurons under constitutive promoters and confirmed non-invasive transduction at targeted brain regions. The non-invasive transduction was achieved using BBB-permeable AAV, PHP.eB, that transduces neurons throughout the central nervous system (CNS) after intravenous delivery. Next, the signals of the markers in the blood were quantified following FUS-BBBO. Lastly, the markers were implemented to measure endogenous neuronal signaling activity. Specifically, they were expressed under the control of a genetic circuit that responds to c-Fos when activated by heightened neuronal activity. The corresponding neuronal activity of the targeted brain regions was measured through blood tests, a process which typically cannot be measured with blood sampling. In this manner, the feasibility of combining genetically-encoded reporters and focused ultrasound to non-invasively and specifically measure endogenous gene expression in the brain is demonstrated.

In one embodiment, a method is provided for measuring gene expression. In accordance with this method, selective genetic expression is caused or promoted of a synthetic marker within a cell type or region of a brain of a subject. Focused ultrasound is selectively applied to a blood-brain barrier of the subject. The engineered marker expressed by the cell type or region of the brain crosses the blood-brain barrier during and/or after application of the focused ultrasound. A blood sample of the subject is obtained. The synthetic marker in the blood sample is quantified using a protein measurement technique. The quantity of the synthetic marker provides a measurement of gene expression.

In a further embodiment, a method is provided for measuring neuronal activity. In accordance with this method, genetic-expression of a marker by neurons is caused or promoted in one or more brain regions of a subject. Focused ultrasound is selectively applied to a blood-brain barrier of the subject. The expressed marker crosses the blood-brain barrier during and/or after application of the focused ultrasound. A blood sample of the subject is obtained. The marker in the blood sample is quantified using a protein measurement technique. The quantity of the marker is indicative of endogenous neuronal signaling activity within the one or more brain regions

In an additional embodiment, a method is provided for determining the success of a gene therapy delivery. In accordance with this method, genetic-expression of a synthetic marker by neurons is caused or promoted in one or more brain regions of a subject. Focused ultrasound is selectively applied to a blood-brain barrier of the subject. The expressed marker crosses the blood-brain barrier during and/or after application of the focused ultrasound. A blood sample of the subject is obtained. The marker in the blood sample is quantified using a protein measurement technique. The quantity of the marker is indicative of endogenous neuronal signaling activity within the one or more brain regions. The success of a gene therapy delivery is determined based on the quantity of the marker in the blood sample.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a schematic view of a technique for non-invasive recovery of marker through insonation, in accordance with aspects of the present techniques;

FIG. 2 depicts a schematic view of blood-brain barrier opening in response to focused ultrasound, in accordance with aspects of the present techniques;

FIG. 3 depicts a schematic view of steps in a study involving expression of a marker after transduction using an adeno-associated viral vector, in accordance with aspects of the present techniques;

FIG. 4 depicts representative images showing subcellular distribution of GLuc after transduction, in accordance with aspects of the present techniques;

FIG. 5 graphically illustrates results of cell-type specificity of the hSyn1 promoter in GLuc expression, in accordance with aspects of the present techniques;

FIG. 6 depicts a schematic view of a technique for evaluating the pharmacokinetics of a marker released from the brain, in accordance with aspects of the present techniques;

FIG. 7 depicts a schematic view of a technique for evaluating marker half-life, in accordance with aspects of the present techniques;

FIG. 8 graphically illustrates a standard curve for a luciferase signal, in accordance with aspects of the present techniques;

FIG. 9 depicts a schematic view of a conditional genetic circuit to tether GLuc expression to neuronal activity, in accordance with aspects of the present techniques;

FIG. 10 depicts a schematic view of a technique for activating neuron AAVs carrying a GLuc expression system, in accordance with aspects of the present techniques; and

FIG. 11 illustrates on- and off-target FUS-BBBO sites in the striatum, in accordance with aspects of the present techniques.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and enterprise-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Gene expression is a critical component of brain physiology, but monitoring this expression in the living brain represents a significant challenge. In particular, monitoring gene expression in the living brain is useful for studying its network activity, diagnosing neurological diseases from onset to progression, and translating gene therapies into the clinic. For example, formation of memories requires activation of gene expression. Gene expression is also implicated in pathogenesis of Alzheimer's disease. Finally, establishing the success of gene therapy delivery is necessary to understand the gene delivery efficacy, durability, and site-specificity. However, mapping brain gene expression poses significant challenges, especially within the brain.

As discussed herein, an approach is taught (referred to herein as Recovery of Markers through InSonation (REMIS)) for non-invasive measurement of gene expression in the brain with cell-type, spatial, and temporal specificity. This approach utilizes engineered protein markers that are produced in neurons but exit into the brain's interstitium. In addition to neurons, other cell types (such as, but not limited to astrocytes, microglia, or oligodendrocytes) can be transduced and/or evaluated for gene expression activity. When ultrasound is applied to targeted brain regions, it opens the blood-brain barrier and releases these markers into the bloodstream. Once in blood, the markers can be readily detected using biochemical techniques. REMIS can non-invasively confirm gene delivery and measure endogenous signaling in specific brain sites through a simple insonation and a subsequent blood test. REMIS is reliable and yields consistent improvement in recovery of markers from the brain into the blood. In this manner, REMIS provides a non-invasive, spatially-specific method of monitoring gene delivery and endogenous signaling in the brain.

With the preceding in mind, the following discussion conveys techniques and results of studies performed using the presently described techniques.

### Secreted Protein Reporter as a Spatially-Specific Marker for Gene Expression in the Brain

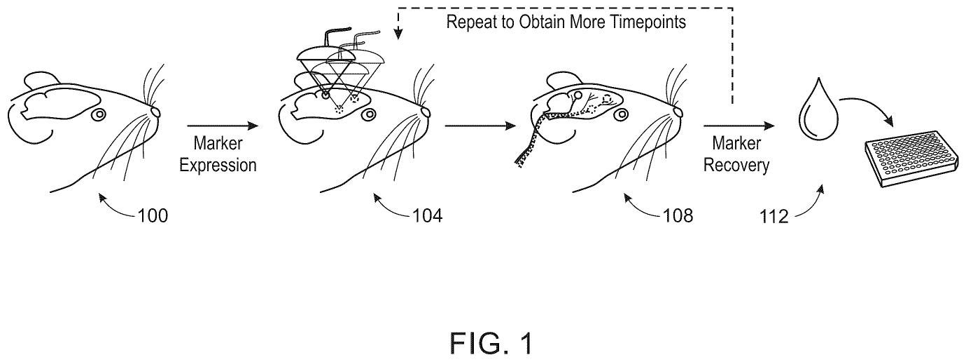

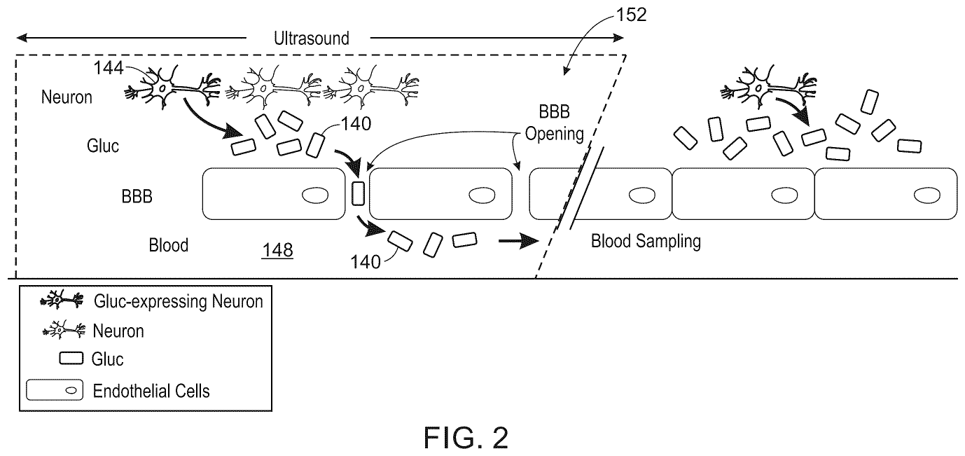

The REMIS platform described herein relies on engineered protein markers that get expressed in cells and are then released into the brain parenchyma. Focused ultrasound (FUS) is used to open the blood-brain barrier (BBB) at specific target sites, allowing the markers to be released into the blood, where they can be detected using biochemical methods. The concept of non-invasive recovery of markers through insonation (REMIS) is illustrated schematically in FIGS. 1 and 2. As shown in FIG. 1, after a baseline blood test and gene delivery (step 100), focused ultrasound is used (step 104) to open the blood-brain barrier (FUS-BBBO) at localized sites and release (step 108) genetically-encoded synthetic markers from the insonated brain regions into the blood. The markers are then collected from simple blood draw for further analysis (step 112) for spatially specific gene expression. In particular, barker levels in the serum can inform on the transgene expression or endogenous physiologic activity in the targeted brain region. As shown in FIG. 1, the post gene delivery steps may be repeated multiple time at different time points to observe changes or trends over time.

As discussed herein, any protein that is secreted from the cell can be used as REMIS marker. With this in mind, and referring to FIG. 2, in the presence of the ultrasound-opened blood-brain barrier, secreted markers 140 produced in transduced cells 144 diffuse into the blood 148. Since ultrasound 152 can be focused with millimeter precision, the markers 140 released into the blood 148 come from a spatially-defined brain region. As depicted in FIG. 2, the leftmost portion of the image (i.e., left of the break) is subject to focused ultrasound, creating gaps in the blood-brain barrier. Conversely, the rightmost portion of the image (i.e., right of the break) is not subject to focused ultrasound and has no corresponding gaps in the blood-brain barrier, leaving the markers behind the blood-brain barrier. Overall, REMIS process enables non-invasive, spatially-specific monitoring of genetically-targeted cells in the brain through a simple blood test. In principle, any serum-detectable protein that can be secreted from cells can be used with REMIS. In the present example illustrated in FIG. 2 Gaussia luciferase (GLuc) was selected for the reporter protein. GLuc is a sensitive reporter that emits bioluminescence through an enzymatic reaction. It is also naturally secreted from the cell as it harbors a cell secretion signal peptide.

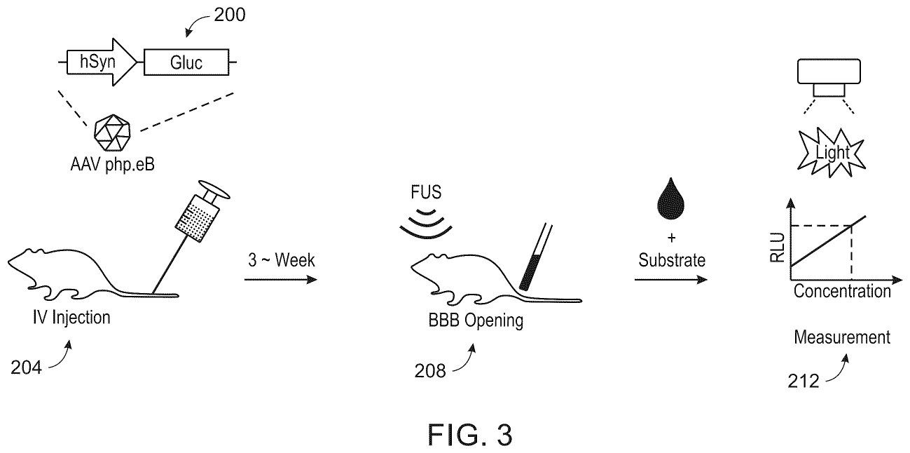

In certain studies discussed herein, to monitor the neuronal activity in specific brain regions, an adeno-associated viral (AAV) vector was constructed that expressed GLuc under the control of the Synapsin 1 (hSyn1) promoter, which drives specific transgene expression in neurons. The hSyn1-GLuc vector 200 (FIG. 3) was delivered to the entire mouse brain using a BBB-permeable, brain-specific AAV serotype called PHP.eB (GLuc-AAV) and subsequent GLuc levels were evaluated in the blood after FUS-BBBO insonation. In this example, mice were intravenously (i.v.) injected (step 204) with GLuc-AAV at 8.3×10⁹ viral particles per g of body weight. Afterwards, GLuc bioluminescence levels were measured while the blood-brain barrier was still intact to obtain baseline GLuc signal levels. Next, the mice were insonated (step 208) with FUS-BBBO using a high peak negative pressure of 0.36 MPa against 8 specific brain regions within the striatum, thalamus, midbrain, and ventral hippocampus. Lastly, blood samples were collected after 7.5 min and bioluminescence readout (step 212) of GLuc levels in the blood obtained.

With respect to this study, the results showed that all GLuc-AAV+ mice had increased GLuc signals in the plasma after insonation, with a mean fold change of 6.1±2.1 (95% confidence interval (CI); P<0.0001, ratio paired t-test; N=9). In the non-transduced control, no changes in GLuc serum levels were observed (P=0.26, ratio paired t-test, N=5).

To confirm BBB opening after insonation, an Evans Blue Dye (EBD) extravasation was performed in the GLuc-AAV+ mice. In particular, representative brain sections on red channel were visualized using EBD extravasation and the targeted regions of the brain approximated. The volume targeted was calculated from the full-width-half-maximum (FWHM) pressure profile, which was approximated as an ovoid with the length of 5.4 mm and width of 0.9 mm, for a total volume of 2.3 mm³. Assuming a C57BL6j mouse brain volume of 508.91 mm³, 8 sites at 2.3 mm³ each would result in 3.6% of the targeted volume. On average, 7.3±0.4 out of 8 targeted brain sites in each mouse showed successful EBD delivery (95% CI; 92% of successful opening 66/72 of the targeted sites, N=9). These regions represented approximately 4% of the brain volume in total. Taken together, these results thus suggest that GLuc is successfully transported from the brain into the blood after insonation.

Although FUS-induced tissue damage typically self-resolves within hours to days and results in no neuronal loss, in additional studies attempts were made to minimize the potential for tissue damage while maintaining successful blood-brain barrier opening and marker release. Such damage typically presents with an extravasation of small numbers red blood cells (RBC) at lower pressures, that can progress to gross damage that is visible in histology, such as vacuolation, or visible hemorrhage when higher pressures are used. Thus, damage defined as the presence of submillimeter-scale pockets of RBCs extravasation that may be observed after FUS-BBBO were the focus of these studies. In particular, this damage was graded based on the size of the bounding ellipse within which all of the damage can be found, and 5 classes of damage were defined: (1) no damage (0 μm), (2) damages below 100 μm, (3) damages between 100-200 μm, (4) damages between 200 and 400 μm, or (5) damages greater than 400 μm. Typically, only a fraction of areas within that bounding box showed any discernible damage. The peak negative pressure levels were chosen to be 0.33 MPa in the striatum, 0.30 MPa in the thalamus and midbrain, and 0.27 MPa in the ventral hippocampus to enable successful opening in at least 7 out of 8 targeted sites in each mouse, while minimizing the presence of damage in the highest damage category. A decision was made to use the lowest tested pressure for thalamus, where damage was present regardless of the tested pressure. Unless otherwise noted, these pressure levels were used for subsequent experiments discussed herein.

Marker release before and after insonation under the optimized pressure levels was investigated and the marker release compared with 0.36 MPa (high) for each brain region. In particular, fold-change in GLuc serum luminescence was assessed before and after FUS insonation with 0.36 MPa peak negative pressure (PNP) (high) or optimized PNP pressure ranging from 0.27 MPa to 0.33 MPa (low) depending on the targeted areas of the brain (P=0.023, two-tailed, unpaired t-test). It was found that lower optimized pressures still released GLuc, though at a lower fold-change over the baseline compared to high pressure (3.0 vs 6.1 arithmetic mean fold-change for low and high pressure, P=0.0023, unpaired two-tailed t-test). However, lower pressure still resulted in highly significant release of the REMIS markers from the brain.

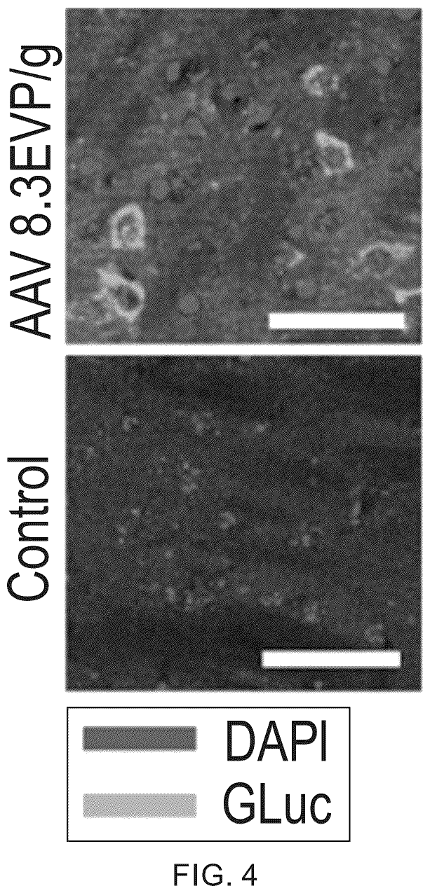

To evaluate whether transgene transduction efficiency affects GLuc signal levels in the serum post-insonation, mice were injected intravenously with AAV PHP.eB carrying GLuc under hSyn1 promoter at different doses (8.3×109, 4.2×109, and 2.1×109 viral particles per g of body weight). After 3-4 weeks of expression, GLuc serum levels were tested before and after insonation. It was observed that, for each dosage group, all mice exhibited enhanced GLuc serum levels after insonation based on the significance of the ratio of change before and after insonation using a ratio paired t-test. The fold-changes compared to baseline were significant for each experiment injected with low, medium, or high dose AAV: 2.6-fold±1.3 (P=0.0044; N=7), 3.7-fold±1.2 (P=0.0002, N=7), and 3.0-fold±1.1 (P=0.0023; N=7) (mean, 95% CI; ratio paired t-test), respectively. Moreover, the ratio of post-insonation GLuc levels to baseline levels was unaffected by viral dose. Further, in a comparison of transduction efficiency as measured by brain histology as the three AAV doses, histologic measurements of brain sections showed that high dose AAV mice resulted in 27.6% GLuc-positive cells, which was significantly higher than values observed with either low and medium viral doses (14.3% and 16.1%, P=0.0009, and P=0.0002, respectively) (P=0.0001; F=15.45; one-way ANOVA, pairwise comparisons P<0.001 when comparing high dose to lower doses, or P>0.05 otherwise.). These results suggest, that ultrasound pressure, rather than the transduction efficiency determines the efficiency of transgene product release from the brain. The absolute levels of post-FUS-BBBO luminescence normalized to mouse weight showed positive correlation of R=0.55 with the transduction levels. Representative images showing transduction of GLuc are shown in FIG. 4, which illustrates representative sections showing subcellular distribution of GLuc immunostaining and nuclear stain DAPI. Scale bar is 50 microns. ns>0.05, *, P<0.05, **, P<0.01, ***, P<0.001, ****, P<0.0001.

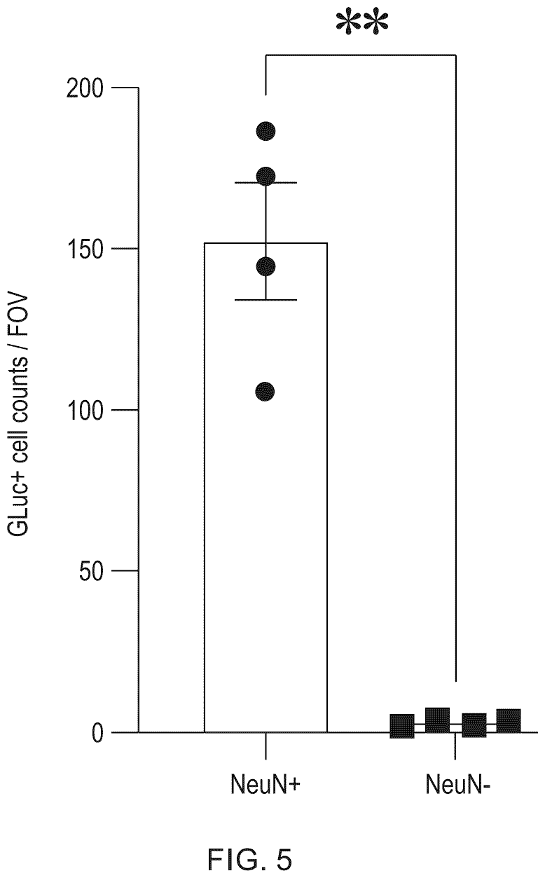

In addition, to confirm cell-type specificity of hSyn1 promoter co-immunostaining was performed for the expression of GLuc marker and the neuronal marker (NeuN) for neurons found within the targeted areas to quantify the number of cells per the field of view (FOV) expressing GLuc that are neurons. It was observed that 98.2%+1.1 (95% CI) of GLuc-transduced cells were neurons, as shown graphically in FIG. 5.

Released Marker Recovery Pharmacokinetics after Insonation

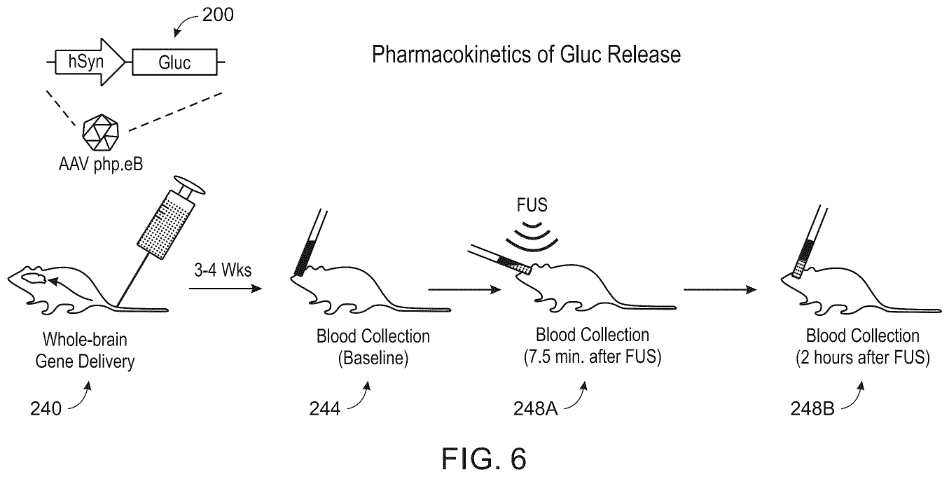

To evaluate the pharmacokinetics of marker released from the brain, and with reference to FIG. 6, blood samples were collected from mice who underwent (step 240) whole-brain gene delivery of GLuc-AAV before (step 244) and after (steps 248A and 248B) insonation of the GLuc-AAV+ mice. GLuc signal levels showed no statistically significant difference between 7.5 min and 120 min after insonation. This result could be explained by several possibilities, including the persistent release of markers from the brain and a marker half-life in the blood that is long compared to the duration of marker release from the brain.

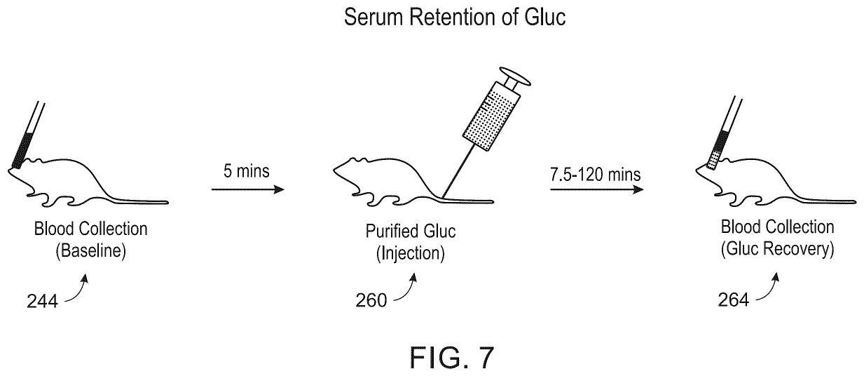

To explore the possibility of a long marker half-life in the blood, a baseline blood collection was initially performed (step 244) on mice. Afterwards, purified GLuc protein was intravenously injected (step 260) into the mice and blood collected (step 264) at different time points from 7.5 to 120 minutes, as shown in FIG. 7. It was observed that the GLuc half-life from a single exponential decay was 7.6±2.4 min (SD; N=30 blood collections in N=15 mice), with over 99.4% of the GLuc eliminated by 60 min after injection. Rather than a long half-life, this result suggests a continuous release of GLuc from the brain over time, with serum levels of GLuc replenishing over 120 min, enabling a broad time window for blood collection for this marker.

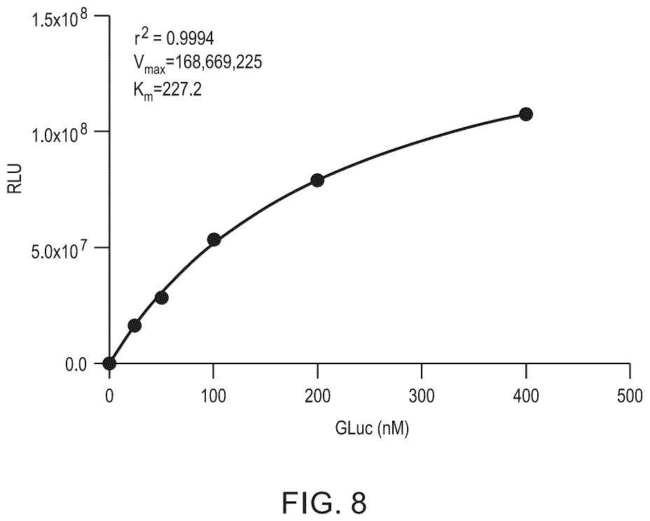

With the understanding that GLuc had a short half-life in serum, a study was performed to evaluate whether the release of markers stops over longer periods of time. A timepoint of 48 hours was selected, which is after a typical blood-brain barrier closure time (e.g., 6-24 h) for FUS-BBBO procedures. A baseline blood collection from GLuc-AAV+ mice was performed. Next, FUS-BBBO was performed and the second blood sample collected within 7.5 minutes. Out of n=6 tested mice, n=5 showed an increase in the serum GLuc following insonation, suggesting a successful FUS-BBBO. Among these 5 mice, a 1.9±0.5-fold (mean with 95% CI, p=0.0068, two-tailed ratio paired t-test) increase was observed in the GLuc levels in the serum following insonation, which was significantly higher than the baseline. After 48 hours, the final blood sample was collected and a significant reduction in the serum marker levels compared to the post-FUS-BBBO peak (2.4±0.3-fold, mcan with 95% CI, p<0.0001, two-tailed ratio-paired t-test) was observed. The GLuc levels at 48 hours were comparable to pre-insonation baseline (0.8±0.2-fold, mean with 95% CI, p=0.14, two-tailed ratio-paired t-test). The absolute levels of GLuc in the serum can be calculated using a standard curve for the luciferase signal using the presently described model and fitted to the Michaclis-Menten model, as shown in FIG. 8. The depicted standard curve for the assay was derived by placing 15 μl of mouse serum in a black 96-well plate and measuring bioluminescence of GLuc by injecting the samples with 50 μl of 20 μM coelenterazine (CTZ) dissolved in luciferase assay buffer using an injector in a microplate reader.

### Non-Invasive Measurement of Neuronal Activity in Specific Brain Regions

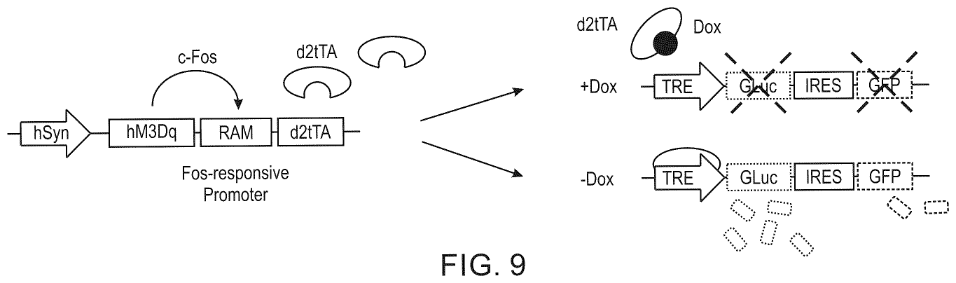

Studies were further performed to determine whether REMIS could be used to measure endogenous neuronal activity in the brain. A conditional genetic circuit, as shown in FIG. 9, was constructed to tether GLuc expression to neuronal activity. To gain temporal control over GLuc, a designer receptor was used that is exclusively activated by designer drug (DREADD) hM3Dq, which induces neuronal firing when turned on by the DREADD agonist clozapine-N-oxide (CNO) back-metabolized to clozapine at subthreshold doses. CNO was selected as the DREADD ligand to take advantage of a multi-hour timeline of drug action following the single administration compared to clozapine. Also incorporated into the circuit was the doxycycline (Dox)-dependent Tet-Off system called Rapid Activity Marking (RAM), which requires both an active c-Fos responsive promoter and the absence of Dox to drive GLuc expression that is then released into the interstitial space of the brain and a nuclearly-localized GFP under internal ribosome entry site (IRES) as a histological ground-truth control of c-Fos activation. To ensure temporal precision of c-Fos recording, the RAM system was shut down with Doxycycline (Dox) until the beginning of recording. As an independent control for measuring c-Fos, GFP was expressed under Internal Ribosome Entry Sites (IRES). Neuronal activity was driven with hM3Dq DREADD. Upon activation, neurons express and release GLuc into the brain interstitium, but express GFP intracellularly.

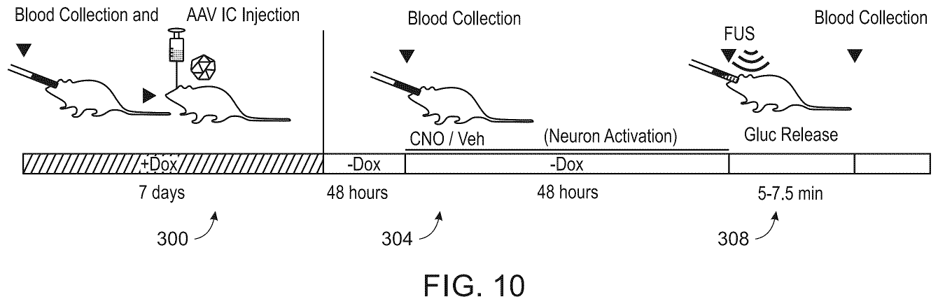

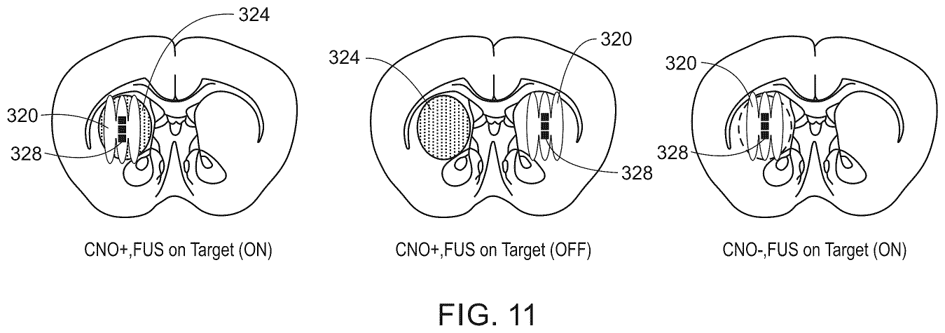

Turning to FIG. 10, after a baseline blood collection (step 244), to activate neurons AAVs carrying the entire Fos-responsive RAM-controlled GLuc expression system were injected (step 300) into the left caudate putamen of mice. Dox was then administered (P.O. in food ad libitum) for 7 d to express RAM system components, while preventing c-Fos-responsive production of GLuc until a specified window needed for neuronal activity recording. Then, 48 h after withdrawing Dox, a baseline blood sample was collected (step 304). Immediately following, Clozapine-N-Oxide (CNO) was injected at a previously-validated dose intraperitoneally (i.p.) to drive neuronal activation and the coupled expression of GLuc. CNO, a prodrug for the DREADD activator in vivo, was chosen over other chemogenetic activators due to its long timeline action, well-suited to long-term recording with the RAM system. As a control, a group of mice were injected with a vehicle control to evaluate baseline levels of GLuc signal in the absence of CNO-induced neuronal activity. After 48 h, the mice were insonated (step 308) at a target site within the striatum (N=10 for vehicle and N=13 for CNO+ mice) (FIG. 11, left and middle). To test the site-specificity of REMIS, a group of CNO+ mice were insonated in the contralateral striatum (N=11) (FIG. 11, right). With this in mind, FIG. 11 illustrates the on- and off-target FUS-BBBO sites 320 in the striatum for each group, activated c-Fos sites 324, and histology measurement sites 328.

Following on-target insonation, it was observed that DREADD-activated neurons in CNO+ mice showed 67.6-fold±44 (mean with 95% CI) increase of GLuc in the blood. This enrichment was higher than that observed in both the vehicle control group (8.3-fold±3.1, mean with 95% CI, P=0.0137, Kruskal-Wallis test, with multiple comparisons) and off-target CNO+ group (8.1-fold±6.3, mean with 95% CI, P=0.0104, Kruskal-Wallis test, with multiple comparisons). Moreover, targeting the contralateral site was not significantly different from targeting the striatum in the vehicle control (P>0.9999, Kruskal-Wallis test), showing the site-specificity of REMIS.

It was then examined whether measurements of neuronal activation using REMIS could be recapitulated histologically. The c-Fos-responsive promoter of the presently described genetic circuit (FIG. 9) is designed to drive concurrent expression of secreted GLuc and intracellular green fluorescent protein (GFP) in response to induced neuronal activity. The data showed that CNO+ mice that expressed GLuc in the striatum had the highest number of GFP-positive cells within the insonated target areas when compared to the off-target mice and vehicle control. Specifically, an 8.0-fold difference in the number of GFP-positive cells between on-target CNO+ mice and the vehicle control (P<0.0001, Kruskal-Wallis test) was observed, which corresponds to the 8.1-fold difference between these two groups when GLuc was measured using REMIS. These results suggest that REMIS can measure c-Fos activation in specific brain regions.

With the preceding studies and discussion in mind, these results establish the suitability of Recovery of Markers through InSonation (REMIS) techniques for non-invasive, site-specific measurement of transgene expression in genetically-targeted cells. In particular, instead of directly measuring gene expression within the deep tissue, which is difficult to achieve, REMIS provides a means of recording reporter concentration in an ultrasound-defined region by transporting engineered markers of gene expression into the blood where they can be easily quantified. These results demonstrate the suitability of this approach for such purposes as confirmation of gene delivery and investigating cellular physiology, including monitoring sustained neuronal activation levels that give rise to c-Fos activity.

As discussed herein, compared to other approaches, REMIS provides various advantages. Traditionally, brain biopsy or post-mortem histology is used to extract cells from the brain and measure gene expression. However, such techniques are invasive and destructive to the tissue. In consequence, they cannot record gene expression activity over multiple timepoints and they damage the very brain region being studied. In contrast, REMIS represents a nondestructive and potentially repeatable alternative to biopsy-based measurement of gene expression.

Further, whereas imaging methods such as MRI with genetically-encoded contrast agents can visualize the entire brain, REMIS enables monitoring of a pre-defined brain region with millimeter precision. REMIS, however, has two advantages. First, it achieves over an order of magnitude higher signal to baseline ratios than observed for genetically-encoded MRI contrast agents. Additionally, REMIS measures molecule concentration through biochemical blood testing, enabling inexpensive detection of multiple types of molecules, possibly in a single sample.

In addition, compared to PET, REMIS does not rely on the development of transgene-binding radioactive BBB-permeable probes, while maintaining comparable spatial precision. When compared to optical imaging, REMIS allows measurement of markers in any brain region that is accessible with focused ultrasound, including behind thick skulls in various species or in deep brain regions both locally or brain-wide. In contrast, for optical methods, such as fluorescence or bioluminescence imaging, depth of penetration and scattering of light through the skull or tissue are technical obstacles. Thus, REMIS has both an advantage over optical methods and future utility in large animal species and deep or large brain regions.

With respect to other techniques, RMAs rely on the site-specific gene delivery to determine the spatial precision and averages signal over the entire transduced cell population. REMIS, conversely, can sample different brain sites within the transduced cell populations, providing it with unique advantages. For example, REMIS can be used for validating gene delivery sites, assessing a diffusion of secreted transgenes, or monitoring gene expression in individual brain sites. Current brain gene therapies struggle with monitoring gene expression without invasive procedures. REMIS offers a non-surgical, non-tissue-destructive solution by augmenting gene therapy to express detectable REMIS markers, akin to using fluorescent proteins in histology. REMIS also could enable long-term spatially-specific transgene expression monitoring, without causing tissue damage, as shown by safety of repeated applications of FUS-BBBO in large animals. The results discussed herein showing non-invasive monitoring of c-Fos in a specific brain region may also be applied to a range of studies. Immediate early genes (IEG) such as c-Fos and Arc are activated by heightened neuronal activity and, thus, REMIS may be used to point to a brain region of interest and measure long-term changes in neuronal activity, for example to determine successful neuromodulation, or activation neuronal ensembles associated with learning and memories.

The spatial resolution of REMIS is dependent upon the parameters of focused ultrasound for the blood-brain barrier opening and ranges from millimeter to sub-millimeter precision. The maximum spatial resolution of REMIS will be defined by the parameters of focused ultrasound and will be equivalent to the volume of opened blood-brain barrier sites.

As discussed in embodiments herein, to ensure that the expression is highly localized, a direct intraparenchymal AAV delivery was selected to express GLuc in unilateral striatum. The studies discussed herein suggest that the spatial diffusion of markers is limited in the tested scenario. c-Fos activation was measured in the striatum contralateral to the area of activation (distance of ˜2.5 mm) and found no diffusion of markers into that region. Moreover, the application of FUS-BBBO to the striatum contralateral to the CNO-activated brain region had an effect that was indistinguishable from opening the BBB in vehicle control mice. This data suggests that extravasation of REMIS markers from the brain is localized within distance on the order of ˜millimeters. The present data also shows that REMIS can be applied after intraparenchymal delivery which is commonly used to achieve spatially-directed AAV delivery in various species. Additionally, the exact time at which the blood is collected after FUS-BBBO may also affect the spatial precision. Over time, proteins from farther away can reach the FUS-BBBO site and escape into the circulation.

The spatial resolution of REMIS could also contribute to variability when measuring absolute levels of brain transduction between individual animals. For example, white matter previously has shown lower level of FUS-BBBO efficacy than the grey matter. Thus, where FUS-BBBO is targeted and how much of the white matter that site contains could affect the absolute levels of REMIS markers released into the serum. To avoid this variability, tracking the gene expression over time within the same site would normalize site-specific effects of REMIS recovery.

REMIS's cell-type specificity is reliant on the genetic targeting. In the results discussed herein, synapsin promoter was used, which restricts expression of the REMIS markers to neurons, and their expression was found to be highly specific. The fold-change of REMIS markers in the serum is dependent on the ultrasound pressure, rather than the viral dose. The process of marker recovery was robust, and it may be noted that every mouse tested in this study showed an increase in the REMIS marker levels after the blood-brain barrier opening, reaching high levels of significance even for lower fold-changes. These results suggest that REMIS may be successfully used in small cohorts of animals, which may be useful in the context of large animal studies or clinical applications.

Further, the temporal resolution of REMIS is largely dependent on the temporal profile of gene expression driving it. The release of markers from the brain itself is rapid with markers appearing in serum within minutes. The c-Fos expression, on the other hand, occurs over minutes to hours timescale, with the RAM system needing 24-48 hours for detectable expression. Importantly for the convenience of measurement a steady, continuous, release of the GLuc was observed over at least 120 minutes. Steady release makes the experimentation less time-dependent and thus more robust. Other markers could be designed that exit the brain more rapidly depending for example on their size and diffusivity, or binding affinity within the extracellular matrix of the brain.

One aspect of note with respect to the presently described technique is the presence of baseline GLuc in the serum even in the absence of blood-brain barrier opening. One possibility is that GLuc is cleared through the glymphatic system and eventually enters the blood. It is also possible that the PHP.eB, while specific to the CNS, may exhibit low levels of peripheral neuron transduction that is sufficient to produce baseline GLuc levels observed. A further possibility is the passage of GLuc through the blood-brain barrier, for example through non-specific interaction of GLuc with FcRN or being encapsulated within the transcytosing endosomes by proximity. Regardless of the mechanism. REMIS reading is independent of the baseline, as it observes changes in serum marker levels over the baseline in response to opening the blood-brain barrier in a specific brain site. This aspect may be of significance in instances where one cannot confirm the origin of the markers in the serum.

As may be appreciated, further improvements, encompassed by the present disclosure, may be obtained via improvements to the various components of the REMIS system: FUS-BBBO, methods of gene-delivery to the brain, gene circuits measuring endogenous gene expression activity, and/or methods of serum marker detection. For example, real-time monitoring of cavitation may be employed with respect to FUS-BBBO to optimize the ultrasound pressures for safety and account for differences in skull attenuation. Similar recovery of markers could also be used for others parts of the CNS such as the eye or spinal cord. Design of new AAV vectors can lead to improved non-invasive delivery. For example, AAVs can be designed to pass through an intact blood-brain barrier after a systemic injection and specifically transduce the central nervous system, or may be more efficiently and tissue-specifically delivered with FUS-BBBO. They can also be optimized to work in different species. These non-invasive gene delivery tools synergize with REMIS which enables similarly non-invasive measurement of the success of gene delivery. Finally, design of new gene circuits, molecular recorders, or mRNA sensors, that can translate cellular physiology into gene expression may enable new applications of non-invasive techniques such as REMIS. REMIS uses biochemical testing which opens up the possibility of measuring multiple types of markers simultaneously, potentially enabling multiplexed monitoring through detection techniques such as mass spectrometry proteomics or single-molecule protein sequencing. Such multiplexed imaging is not currently possible with MRI, PET, or with optical methods, where the number of colors in in vivo imaging is orders of magnitude fewer than what can be detected through serum sampling with proteomic techniques. Such improvements may further facilitate the development and translation of REMIS as a paradigm for precise non-invasive monitoring of genetically-targeted cell populations within specific brain regions.

### Methods

By way of further developing the techniques discussed herein, a brief discussion of methodology is provided for completeness.

Animals—Wild-type C57BL/6J (Strain #000664) male and female mice were obtained from Jackson Laboratory (Bar Harbor, ME). Animals were housed in a 12 h light-dark cycle and were provided with food and water ad libitum. Mice were selected randomly for experiments, ensuring that each cage was represented in multiple comparison groups to avoid cage-specific effects. All mice for which it was possible to obtain sufficient amount of blood or tissues were included in the analysis. All experiments were conducted under a protocol (#IACUC-23-138-RU) approved by the Institutional Animal Care and Use Committee of the Rice University and ARRIVE guidelines were followed during the reporting.

Plasmid Construction—To construct AAV-hSyn-GLuc, the vector AAV-hSyn-GLucM23-iChloC-EYEP (Addgene #114102) was digested with Spel and EcoRV (New England Biolabs) to isolate the backbone containing the hSyn promoter. GLucM23, a GLuc variant, was amplified by PCR from the same vector and its DNA was extracted using the Monarch DNA Gel Extraction Kit (New England Biolabs, Ipswich, MA). GLuc was inserted into the digested backbone through Gibson Assembly Hi-Fi kit (New England Biolabs, Ipswich, MA). AAV-hSyn-hM3Dq-RAM-d2tTA was constructed using previously described techniques. To construct AAV-RAM-GLuc-IRES-GFP, the GLuc DNA was amplified from an AAV-TRE-RMA-IRES-GFP plasmid and assembled into the same backbone after digestion using Pmel and BamHI. AAV-hSyn-CLuc was constructed similarly by amplifying the CLuc DNA from CLuc-RMA (CLuc-Fc) plasmid and inserted into the same backbone after digestion using EcoRV and BamHI.

Adeno-associated virus production—PHP.cB AAV virus was packaged with the AAV-hSyn-GLuc plasmid construct using a commercial service (Vector Builder, Chicago, IL) and the titer was provided by the manufacturer.

Intravenous administration of AAVs—AAV was injected intravenously (i.v.). Mice at 10-14-weeks old were anesthetized with 2% isoflurane in oxygen and then cannulated in the tail vein using a 30-gauge needle connected to PE10 tubing. The cannula was then flushed with 10 units (U) ml⁻¹ of heparin in sterile saline and affixed to the mouse tail using a tissue adhesive. Subsequently, the mice were injected via tail vein with PHP.eB AAV (2.1-8.3×10⁹ viral particles per g of body weight) encoding GLuc under the hSyn promoter. AAV injected mice were housed for 3-4 weeks to allow for gene expression.

Focused Ultrasound BBB opening—Mice at 14-18-weeks old were anesthetized with 1-2% isoflurane in oxygen. The hair on mice head was removed by shaving with a trimmer. The mice were then cannulated in the tail. Subsequently, the mice were placed on a stereotaxic mount, with their heads held in place with a custom-made plastic nosecone and secured with car bars. Bregma and Lambda markers were used to target ultrasound to specific brain regions using a stereotaxic frame (RK-50, FUS Instruments). An ultrasonic transducer was coupled to the shaved area of the head via degassed water in water bath and degassed aquasonic ultrasound gel. The mice were injected with approximately 2.8×10⁶ DEFINITY microbubbles (Lantheus) dissolved in sterile saline, per g of body weight for each targeted site. For each targeted site, DEFINITY was re-injected before insonation. The ultrasonic parameters used were 1.5 MHz, 10 ms pulse train length, 1 Hz pulse repetition frequency for 120 pulses. The pressure for insonation was varied between experiments and targeted sites based on evaluation of safety and efficacy of blood-brain barrier opening in this study. The range of peak-negative-pressure used in this study was 0.27-0.36 MPa, as calibrated by the manufacturer (FUS Instruments, Toronto, Canada). In the GLuc recovery experiment, targeted sites represented 4% of the brain volume in total, as measured by full-width half-maximum pressure of the FUS field. Due to equipment failure, experiments on c-Fos measurement used a new transducer with a different calibration curve. Blood-brain barrier opening and tissue damage were used to confirm the validity of new calibration in vivo and used manufacturer-calibrated pressure of 0.46 MPa, which is not directly comparable with the pressures used in previous experiments due to differences in manufacturer's (FUS instruments, Toronto, Canada) calibration methods. However, the opening was present and comparable to previous experiments with 91.2% (31/34) mice showing opening and no apparent damage was observed in N=12 randomly selected mice (N=4 per group for CNO with on target FUS-BBBO, CNO with off target FUS-BBBO, and vehicle with on-target FUS-BBBO). After ultrasound insonation, aquasonic ultrasound gel was removed and the incision was closed. Evans Blue Dye (5%) dissolved in 1×PBS was injected intravenously. EBD passes selectively through a permeable blood-brain barrier and localizes in the brain parenchyma, allowing visualization of where the blood-brain barrier has been opened. Successful blood-brain barrier opening showing EBD extravasation was a criterion for inclusion in the FUS-BBBO groups, except for mice in the study of non-invasive measurement of c-Fos in specific brain regions with REMIS, where histology to assess blood-brain barrier opening was not possible due to terminal blood collection occurring after the expected blood-brain barrier closure. After 20 min, mice were perfused with 10% neutral buffered formalin (Sigma-Aldrich) after displacing blood with 10 units (U) ml⁻¹ of heparin in 1×PBS via cardiac perfusion.

Pharmacokinetic analysis of GLuc serum half-life—Purified GLuc protein (Nanolight Technology) was injected intravenously through a tail vein catheter. C57B16j mice at 10-14-weeks old were injected with purified GLuc protein in IX TBS under isoflurane anesthesia (1-5% in oxygen). After a period of 7.5-120 minutes, mice were again anesthetized in 2% isoflurane in oxygen. Afterwards, 1-2 drops of 0.5% ophthalmic proparacaine were applied topically to the cornea of an eye and a heparin-coated microhematocrit capillary tube (Fisher Scientific) was placed into the medial canthus of the eye and the retro-orbital plexus was punctured to withdraw 50 μl of blood. Each mouse underwent a maximum of three blood collections-baseline measurement and measurement at 7.5 minutes followed by one additional collection of which the last was terminal. The collected blood was centrifuged at 5,000 g for 15 min to isolate plasma and stored at −20° C. until use.

Luciferase assay—To conduct luciferase assay, 15 μl of plasma was placed in a black 96-well plate. Bioluminescence of GLuc was measured by injecting plasma samples with 50 μl of 20 UM coelenterazine (CTZ) (Nanolight Technology) dissolved in luciferase assay buffer using an injector in a Tecan M200 microplate reader (Männedorf, Switzerland). The luciferase assay was calibrated to the standard curve with purified GLuc protein (Nanolight Technology), using the same protocols as in the pharmacokinetics analysis, in mouse serum (GeneTex).

Immunostaining—Mice brains were extracted and postfixed in 10% neutral buffered formalin overnight at 4° C. Coronal sections were cut at a thickness of 50 μm using a vibratome (Leica) and stored at 4° C. in 1×PBS. Sections were stained as follows: 1) pre-incubate with permeabilizing agent (1% Triton X-100, 0.5% Tween 20 in 1×PBS) for 1 h; 2) block for 1 h at room temperature with blocking buffer (0.3% Triton X-100 and 10% normal donkey serum in 1×PBS); 3) incubate with primary antibody overnight at 4° C.; 4) wash in 1×PBS for 10 min 3 times; and 5) incubate with secondary antibody for 4 h at room temperature. After last washes in 1×PBS, sections were mounted on glass slides using the mounting medium (Vector Laboratories) with or without DAPI and cured overnight in dark at room temperature. Antibodies and dilutions used are as follows: rabbit anti-GLuc (1:1,500, Nanolight Technology) and Alexa 488 secondary antibody (1:500, Life Technologies). All images were acquired by the BZ-X810 fluorescence microscope (Keyence).

Statistical analysis—Two-tailed paired t-test with unequal variance was used to compare two data sets when comparing means, or ratio-paired t-test was used to compare fold changes in serum marker levels with FUS-BBBO marker recovery. One-way ANOVA or Kruskal-Wallis tests with Tukey honestly significant difference post hoc test was used to compare means between more than two data sets in any other case, depending on whether the standard deviations of compared groups were significantly different in Brown-Forsythe test. Kruskal-Wallis was selected for c-Fos measurement data due to showing significantly non-normal distribution (Shapiro-Wilk test for CNO-on-target FUS group, P=0.0006). All P values were determined using Prism (GraphPad Software), with the statistical significance represented as ns (not significant), *P<0.05, **P<0.01, ***P<0.001, ****P<0.0001. The sample and effect sizes were calculated using G*power 3 software, assuming 80% power, and alpha value of 0.05. In the absence of previous data that could be used for calculation of effect size, sample sizes used in invasive measurement of tissue gene expression were relied on, or in non-invasive chemogenetic neuromodulation with gene delivery using FUS-BBBO.

Stereotaxic injection of AAV—AAV were injected into the brains using a microliter syringe equipped with a 34-gauge beveled needle (Hamilton) installed to a motorized pump (World Precision Instruments) using a stereotaxic frame (Kopf). For AAV, PHP.cB serotype was used. For chemogenetic neuromodulation experiments, three different AAVs were delivered. AAV encoding hSyn-hM3Dq-RAM-d2tTA and RAM-GLuc-IRES-GFP were delivered to the left hemisphere caudate putamen (CPu) in the striatum in the following ipsilateral coordinates: (AP +1.0 mm, ML +2.0, DV +3.8 mm), (AP +0.5 mm, ML +1.5, DV +3.0 mm), (AP −0.5 mm, ML +2.5, DV +3.5 mm). AAV encoding hSyn-CLuc was delivered to the right hemisphere CPu in the striatum in the contralateral coordinates. For each mouse, 1.0×10⁹ viral particles of each AAVs were delivered correspondingly. For AAV injections, 568 nl AAV (hSyn-hM3Dq-RAM-d2tTA) and 116 nl AAV (RAM-GLuc-IRES-GFP) were prepared in a cocktail and 684 nl total volume was injected each site. AAV (hSyn-CLuc) alone was injected 500 nl each site. The injection speed was 250 nl min⁻¹. The needle was kept at the injection site for 10 min owing to the relatively high volume of injection.

Doxycycline control of gene expression—For chemogenetic neuromodulation experiments, mice were placed on 40 mg kg⁻¹ of Dox chow (Bio-Serv) 24 h prior to intracranial injection of AAVs. Dox chow was maintained for 7 d after the AAV injection. Dox chow was removed 48 h prior to inducing chemogenetic activation.

Chemogenetic activation of neuronal activity-Water-soluble CNO (Hello Bio #HB6149) was dissolved in saline (Hospira) at 1 mg ml⁻¹ and stored at −20° C. until use. To induce chemogenetic activation of mice expressing hM3Dq, CNO was injected intraperitoneally (i.p.) at 5 μg per g mouse, respectively.

Blood collection for luciferase assay—For the groups of mice in GLuc in vivo half-life measurement experiment, baselines were collected 5 min prior to GLuc i.v. injection. Experimental samples were collected 7.5 min post-injection in common and 30-, 60-, and 120-min post-injection based on their assigned groups. Up to 50 μl of blood was collected for each timepoint, and each mouse underwent a maximum of three blood collections, including the baseline measurement. For the groups of mice measuring transduction after systemic AAV delivery, baselines were collected before subcutaneous analgesic drug injection in the ultrasound insonation procedure. Experimental samples were collected 7.5 min after an ultrasound insonation session. For the group of mice in GLuc recovery in extended time frame experiments, along with baseline measurement, experimental samples were collected twice, 7.5 min and 120 min after an ultrasound session, 50 μl in volume per collection. For the groups of mice where we used REMIS to measure chemogenetic activation, baselines were collected twice; before intracranial AAV injection and before CNO i.p. administration. Experimental samples were collected 7.5 min after an ultrasound insonation session. Up to 100 microliters of blood was collected for each sample, with the last sample always being a terminal collection, followed by immediate cardiac perfusion.

Hematoxylin staining—Sectioned brain samples were stored in 1×PBS and prepared in water 10 min prior to staining. Samples were dehydrated in 100% ethanol and 95% ethanol, 1 min each. Then, the samples were rinsed in water for 1 min. Next, samples were immersed in 0.4× diluted Mayer's Modified Hematoxylin solution (Abcam, #64795) for 1 min and rinsed again in water for 3 min. Nuclear stain was completed by immersing samples in the Blueing reagent (Abcam, #66152) for 20 sec. Finally, samples were rinsed in water for another 3 min. Samples were rehydrated in water for another 10 min before mounting.

Cell positivity quantification—C-Fos images were taken on a Keyence BZ-x810 using the 20× objective lens. Each brain section had three images taken, one on top of the other in a vertical line inside of the blood brain barrier opening guided by the EBD signal. Images were counted manually in Zen Software (J.S.T.). Each image was loaded in and the upper limit of the histogram was reduced to 15,000 out 65536 for every image to aid in visualization of all cells. Blinding was not possible due to the stark differences between the experimental group and controls.

GLuc cell counts images were taken on a Keyence BZ-X810 using the 20× objective lens. Single hemisphere images were taken and counted in Zen software by adding a horizontal only grid line spaced by (400 pixels) vertically between each line. Only green and blue cells that touched this line were counted. Green cells were taken in the 488 channel and designate GLuc expression. Blue cells were taken in the DAPI channel and were stained using DAPI containing mounting media. The experimenter was blinded to identity of the groups.

Cell specificity for GLuc and NeuN positive images were taken on a Zeiss LSM-800 Confocal microscope. NeuN was stained using Novus Biologicals RBFOX3/NeuN antibody conjugated to Alexa Fluor 405. GLuc stained with anti-GLuc antibody (nanolight technology) and an Alexa Fluor 594 secondary antibody. GLuc and NeuN images were taken using the 20× objective lens on the confocal, each image was overlaid and counted manually using Zen software. Total of 11 sections across 4 mice was stained and counted (2-3 sections per mouse).

With the preceding methodology details and study results in mind, it may be appreciated that a technical improvement of the present disclosure is the use of synthetic serum markers to monitor transgene expression in specific, localized brain regions through ultrasound-triggered release. By way of comparison to other approaches and related techniques, the present technique uses focused ultrasound, as opposed to reverse transcytosis or comparable techniques, to facilitate passage of an engineered (e.g., designed) protein marker across the blood-brain barrier, such as to allow passage of markers to the blood where they might be sampled using a conventional blood draw.

Further, there is an as yet unmet need in the area of central nervous system (CNS) diseases due to the lack of fast and sensitive diagnostic methods and effective treatments. CNS biomarkers are one tool in the identification of patients at risk, early diagnosis, follow-up of disease progression, and effectiveness of treatments in neurology, in particular for demyelinating diseases (such as multiple sclerosis), neurodegenerative diseases (such as Alzheimer's disease) or traumatic brain injury. The present techniques offer opportunities for addressing the need for diagnostic methods and treatment development in this field. In particular, in order to expedite drug development for neurology diseases, it is important to identify novel biomarkers that enhance diagnostic and prognostic accuracy, improve the existing decision criteria for early diagnosis and risk stratification, assist in disease monitoring, and act as surrogate endpoints in experimental studies and clinical trials. The present techniques provide one tool in expediting such development.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims

What is claimed is:

1. A method for measuring gene expression, the method comprising:
causing selective genetic expression of a synthetic marker within a cell type or region of a brain of a subject;
selectively applying focused ultrasound to a blood-brain barrier of the subject, wherein the engineered marker expressed by the cell type or region of the brain crosses the blood-brain barrier during and/or after application of the focused ultrasound;
obtaining a blood sample of the subject; and
quantifying the synthetic marker in the blood sample using a protein measurement technique, wherein the quantity of the synthetic marker provides a measurement of gene expression.

2. The method of claim 1, wherein the cell type comprises neurons, astrocytes, microglia, and oligodendrocytes.

3. The method of claim 1, wherein the synthetic marker comprises a secreted protein.

4. The method of claim 1, wherein the protein measurement technique comprises one of single molecule array for protein detection (SIMOA), enzymatic activity assays specific to the test protein, SDS-page, western blot, next-generation protein sequencing, enzyme-linked immunosorbent assay (ELISA), in vivo detection of luciferases, surface plasmon resonance (SPR), or mass spectrometry.

5. The method of claim 1, further comprising diagnosing a neurological disease based on the quantity of the synthetic marker.

6. The method of claim 1, further comprising determining the success of a treatment based on the quantity of the synthetic marker.

7. The method of claim 6, wherein the treatment comprises a gene therapy delivery and the quantity of the synthetic marker in the blood is used to determine one or more of gene delivery efficacy, durability, or site-specificity.

8. A method for measuring neuronal activity, the method comprising:
causing genetic-expression of a marker by neurons in one or more brain regions of a subject;
selectively applying focused ultrasound to a blood-brain barrier of the subject, wherein the expressed marker crosses the blood-brain barrier during and/or after application of the focused ultrasound;
obtaining a blood sample of the subject; and
quantifying the marker in the blood sample using a protein measurement technique, wherein the quantity of the marker is indicative of endogenous neuronal signaling activity within the one or more brain regions.

9. The method of claim 8, wherein the marker comprises a synthetic marker transduced into the one or more brain regions.

10. The method of claim 9, wherein the synthetic marker is transduced using one of an adeno-associated (AAV) vector, mRNA lipids nanoparticles (LNPs), lentivirus, or other synthetic nanoparticles.

11. The method of claim 8, wherein causing genetic-expression of the marker comprises causing expression of the marker under the control of a genetic circuit that responds to c-Fos when activated by heightened neuronal activity.

12. The method of claim 8, wherein the protein measurement technique comprises one of single molecule array for protein detection (SIMOA), enzymatic activity assays specific to the test protein, SDS-page, western blot, next-generation protein sequencing, enzyme-linked immunosorbent assay (ELISA), in vivo detection of luciferases, surface plasmon resonance (SPR), or mass spectrometry.

13. The method of claim 8, further comprising diagnosing a neurological disease based on the quantity of the marker.

14. The method of claim 8, further comprising determining the success of a gene therapy delivery based on the quantity of the marker in the blood sample.

15. The method of claim 14, wherein the quantity of the synthetic marker in the blood is used to determine one or more of gene delivery efficacy, durability, or site-specificity.

16. A method for determining the success of a gene therapy delivery, the method comprising:
causing genetic-expression of a synthetic marker by neurons in one or more brain regions of a subject;
selectively applying focused ultrasound to a blood-brain barrier of the subject, wherein the expressed marker crosses the blood-brain barrier during and/or after application of the focused ultrasound;
obtaining a blood sample of the subject;
quantifying the marker in the blood sample using a protein measurement technique, wherein the quantity of the marker is indicative of endogenous neuronal signaling activity within the one or more brain regions; and
determining the success of a gene therapy delivery based on the quantity of the marker in the blood sample.

17. The method of claim 16, wherein causing genetic-expression of the marker comprises causing expression of the marker under the control of a genetic circuit that responds to c-Fos when activated by heightened neuronal activity.

18. The method of claim 16, wherein the quantity of the synthetic marker in the blood is used to determine one or more of gene delivery efficacy, durability, or site-specificity of the gene therapy delivery.

19. The method of claim 16, wherein the protein measurement technique comprises one of single molecule array for protein detection (SIMOA), enzymatic activity assays specific to the test protein, SDS-page, western blot, next-generation protein sequencing, enzyme-linked immunosorbent assay (ELISA), in vivo detection of luciferases, surface plasmon resonance (SPR), or mass spectrometry.

20. The method of claim 16, further comprising transducing the synthetic marker into the one or more brain regions.
